(19) United States
(12) Patent Application Publication
(10) Pub. No.: US 2012/0100125 A1
(43) Pub. Date: Apr. 26, 2012

(54) LOCAL DEHYDRATION FOR CARPAL TUNNEL SYNDROME

(75) Inventor: Elaine Lee, Santa Clara, CA (US)

(73) Assignee: KYPHON SARL, Neuchatel (CH)

(21) Appl. No.: 12/912,594

(22) Filed: Oct. 26, 2010

(51) Int. Cl. A61K 38/51; A61P 25/02; A61K 38/47; A61K 38/02; A61M 31/00; A61K 38/48

(52) U.S. Cl. 424/94.5; 604/57; 424/94.67; 424/94.62; 514/17.7

(57) ABSTRACT

A procedure for treating carpal tunnel syndrome can involve delivering a dehydrating agent into and/or near the carpal tunnel. The dehydrating agent can reduce swelling of the flexor tendons and/or the carpal ligament in the region, thereby reducing pressure on the median nerve that also runs through the carpal tunnel. Through the use of a dehydrating agent that affects proteoglycans without affecting collagen, localized dehydration can be induced without weakening the flexor tendons, carpal ligament, and any other structures in the carpal tunnel region.

### FIELD OF THE INVENTION

The invention relates to a system and method for minimally invasive surgical treatment of carpal tunnel syndrome.

### BACKGROUND OF THE INVENTION

The carpal tunnel is an opening through the wrist into the hand that is formed by the carpal bones of the wrist on the bottom and the transverse carnal ligament on the top. The transverse carpal ligament is at the base of the wrist and crosses from one side of the wrist to the other. It is sometimes referred to as the carpal ligament or the flexor retinaculum.

The median nerve and flexor tendons of the hand run through the carpal tunnel. The median nerve rests on top of the flexor tendons, just below the carpal ligament. It gives sensation to the thumb, index finger, long finger, and half of the ring finger. It also sends a nerve branch to control the muscles of the thumb.

In general, carpal tunnel syndrome (CTS) develops when the tissues around the median nerve swell and press on the nerve. Early in the disorder, the process is reversible. Over time, however, the insulation on the nerves may wear away, and permanent nerve damage and severe loss of hand function may develop, along with pain, numbness, and tingling in the wrist, hand, and fingers. Only the little finger is unaffected by the median nerve.

Virtually all workers who use their hands and wrists repetitively are at risk for CTS, particularly if they work in cold temperatures and have factors or medical conditions that make them susceptible. For example, computer users/typists, workers in the meat and fish packing industries, airplane assemblers, and musicians are among those at very high risk for CTS. In addition, people who intensively cook, knit, sew, do needlepoint, play computer games, do carpentry, or extensively use power tools are likewise at increased risk for CTS.

Ideally, the early phases of carpal tunnel syndrome are treated before the damage progresses. A conservative approach to CTS, which may include corticosteroid injections and splinting, is typically the first step in treating this disorder. The conservative approach is most successful in patients with mild carpal tunnel syndrome. A concurrent regimen of physical therapy (e.g., a program of hand/wrist stretching and strengthening) may provide further benefits. In addition, alternative therapies such as ultrasound, nonsteroidal anti-inflammatory drugs (NSAIDs), ice/warmth, low-level laser therapy (LLLT), dietary modification, oral diuretics, and acupuncture have been used in the treatment of CTS, with varying degrees of success.

For severe cases of CTS, surgery often proves to be a more effective treatment option. Surgery is also more likely to be necessary for patients with underlying conditions such as diabetes. Even among patients with mild CTS, there is a high risk of relapse. Some researchers are reporting better results when specific exercises for carpal tunnel syndrome are added to the program of treatments.

Traditionally, in a CTS surgery, the carpal ligament is cut free (“released”) from the median nerve, thereby relieving the pressure on the median nerve. The most common approach has been an open surgical procedure (“open carpal tunnel release” or “open release”) performed in an outpatient facility, and is a straightforward and well-characterized procedure.

In recent years, more surgeons have adopted a “mini” open—also called short-incision-procedure. This surgery requires only a one-inch incision, but it still allows a direct view of the area (unlike endoscopy, which is viewed on a monitor). The mini-open approach may allow for quicker recovery while avoiding some of the complications of endoscopy, although few studies have investigated its benefits and risks. The recovery time in patients receiving the mini-open approach may be shorter than with the open approach, and results are generally the same.

Endoscopy for CTS is another less invasive procedure than standard open release. In an endoscopic release procedure, a surgeon makes one or two ½-inch incisions in the wrist and palm, and inserts one or two endoscopes (pencil-thin tubes). The surgeon then inserts a tiny camera and a knife through the lighted tubes. While observing the underside of the carpal ligament on a screen, the surgeon cuts the ligament to free the compressed median nerve.

Endoscopic release patients report less pain than those who had the open release procedure, and return to normal activities in about half the time. Nevertheless, at this time the best evidence available does not show any significant long-term advantages of endoscopy over open release in terms of muscle, grip strength, or dexterity. The endoscopic approach may even carry a slightly higher risk of pain afterward. This may be due to a more limited view of the hand with endoscopy. Concerns of irreversible nerve injury with endoscopic carnal tunnel release, when compared with open carpal tunnel release, exist because of this reduced visibility.

Regardless of the procedure, patients who have undergone conventional CTS surgery typically report some permanent loss of grip strength, a loss of lifting strength in the wrist/forearm and nagging loss of lull range of motion of the hand and wrist alter surgery. This is due to the severing of the carpal ligament. The purpose of the carpal ligament is to wrap around the hand and wrist and hold the many small bones of the hand and wrist securely together. The carpal ligament also maintains the flexor tendons in a path that enables the mechanical advantage necessary for strong grip strength and range of motion. These support capabilities are naturally diminished when the ligament is severed.

Accordingly, it is desirable to provide a system and technique for alleviating the symptoms of CTS while maintaining as much grip strength and/or range of motion of the hand and wrist.

### SUMMARY OF THE INVENTION

By delivering a dehydrating agent directly to the carpal tunnel tissues, local swelling can be reduced, thereby reducing pressure on the median nerve and mitigating the symptoms of carpal tunnel syndrome (CTS). Because the dehydrating agent can be delivered via a needle, the procedure can be performed in a minimally invasive manner while still providing immediate, and potentially long-term, relief from the symptoms of CTS.

A procedure for treating CTS can involve locally delivering a dehydrating agent to the carpal tunnel region. This delivery can be performed in a minimally invasive fashion (e.g., using needles, shunts, patches, or compressed gas injection systems), or via mini-open or open procedures. For example, in some embodiments, one or more needles can be used to inject the dehydrating agent into or around swollen flexor tendons and/or the carpal ligament to dehydrate those elements and reduce compression of the median nerve.

In various other embodiments, alternative delivery mechanisms can be used to convey the dehydrating agent to the carpal tunnel region. For example, controlled delivery systems (e.g., pumps, patches, or timed release capsules) can be used to deliver the dehydrating agent at a desired rate, frequency, or duration to the carpal tunnel region.

In some embodiments, a dehydrating agent that affects proteoglycans but not collagen can be used to reduce swelling of the flexor tendons and/or the carpal ligament while minimizing any effects on the structural integrity of the flexor tendons, carpal ligament, and/or adjacent anatomical elements. Such proteoglycan-targeted dehydrating agents can include enzymes such as chondroitinase ABC, matrix metalloproteinase-3, and hyaluronidase, among others, and peptides such as polylysine.

In various other embodiments, the localized dehydrating agent delivery can be combined with localized therapeutic substance delivery to the carpal tunnel region to augment, enhance, and/or broaden the range of CTS symptom relief. For example, anti-inflammatory drugs, antibiotics, corticosteroids, pain medications, analgesics, anesthetics, relaxants, enzymes, lubricants, anti-fibrotic agents, growth factors, nucleic acids, cells, and/or other therapeutic agents could be delivered to the flexor tendons, the transverse carpal ligament, and/or anywhere else in the general vicinity of the carpal tunnel. In some embodiments, the therapeutic substance delivery can be through the same delivery system(s) (e.g., the same needle(s)) used for delivery of the dehydrating agent. In other embodiments, dedicated delivery structures (e.g., needles) can be used for the therapeutic substance delivery.

A surgical kit for treating CTS can include a delivery system (e.g., one or more needles, shunts, syringes, pumps, implants, patches, etc.) and instructions for using the delivery system for delivering a dehydrating agent to the carpal tunnel region. In some embodiments, the kit can further include a supply of the dehydrating agent, either in a discrete storage container (e.g., a vial(s) or ampoule(s)) or preloaded into the delivery system. In various other embodiments, the kit can further include a secondary delivery system and/or instructions for delivering a therapeutic substance to the carpal tunnel region.

As will be realized by those of skilled in the art, many different embodiments of a method for treating CTS using local dehydration and optional therapeutic substance delivery, and/or a surgical kit for local dehydration of the carpal tunnel region are possible. Additional uses, advantages, and features of the invention are set forth in the illustrative embodiments discussed in the detailed description herein and will become more apparent to those skilled in the art upon examination of the following.

### BRIEF DESCRIPTION OF THE DRAWINGS

FIGS. 1A-1B show an example of median nerve compression due to tendon swelling in the carpal tunnel.

FIG. 1C shows an exemplary localized dehydration treatment for the carpal tunnel region.

FIG. 1D shows an exemplary result of localized dehydration within the carpal tunnel.

FIG. 2 shows a kit including a delivery system for delivering a dehydration agent to the carpal tunnel region and instructions for use of the delivery system.

### DETAILED DESCRIPTION

By delivering a dehydrating agent directly to the carpal tunnel tissues, local swelling can be reduced, thereby reducing pressure on the median nerve and mitigating the symptoms of carpal tunnel syndrome (CTS). Because the dehydrating agent can be delivered via a needle, the procedure can be performed in a minimally invasive manner while still providing immediate, and potentially long-term, relief from the symptoms of CTS.

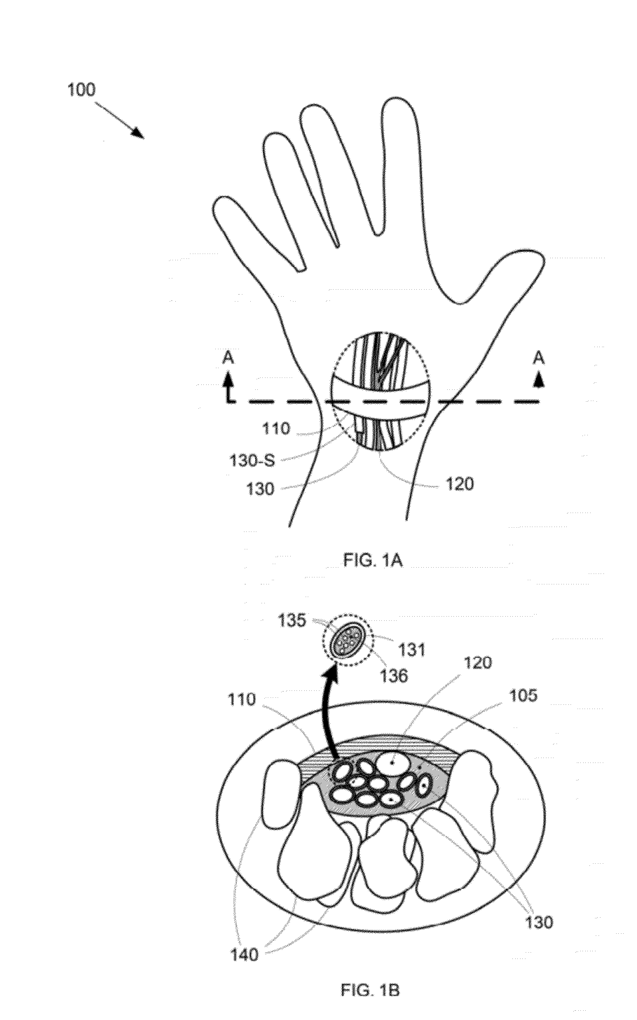

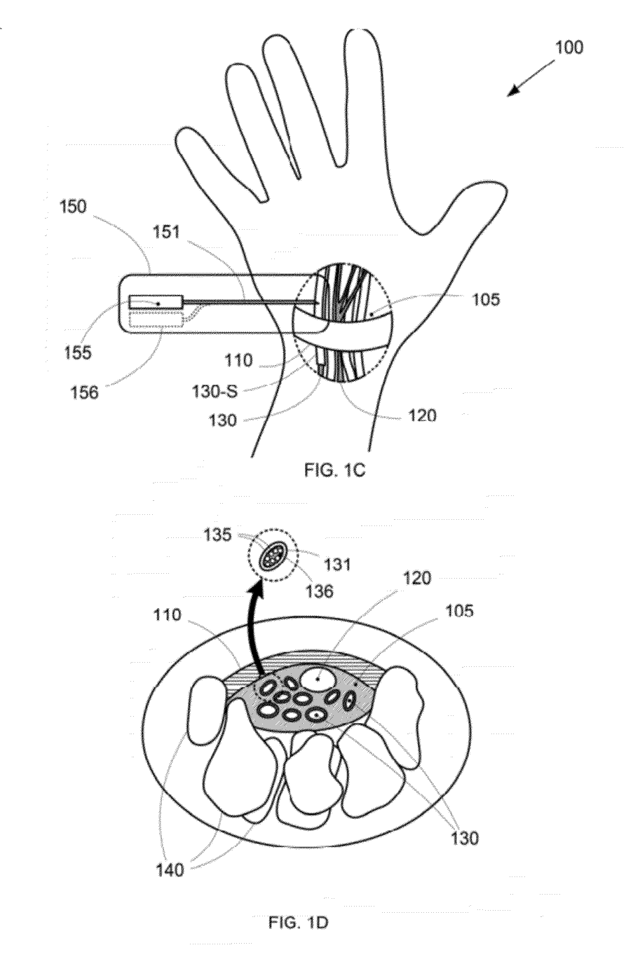

FIGS. 1A-1D show an exemplary procedure involving local dehydration in the carpal tunnel region for the treatment of CTS. In FIG. 1A, a cutaway view is shown of a hand 100 that shows the position of the median nerve 120 running beneath the transverse carpal ligament 110, along with the surrounding flexor tendons 130 that bend the fingers and thumb of hand 100.

Note that hand 100 would typically be the hand of a patient suffering from CTS, with the below-described procedure being an exemplary treatment of that patient. However, in various other embodiments, hand 100 could be the hand of a cadaver, or simply a model hand, with the below-described procedure being performed for training/instructional purposes.

Sectional view A-A shown in FIG. 1B depicts the carpal tunnel 105 formed by the carpal ligament 110 and the wrist (carpal) bones 140. Flexor tendons 130 and median nerve 120 are all packed into the relatively narrow carpal tunnel 105, and therefore any swelling of flexor tendons 130 and/or carpal ligament 110 can result in problematic compression of median nerve 120.

As indicated in the detail view of tendon 130 in FIG. 1B, each tendon is formed of a bundle of collagen fibers 135 supported in an extracellular matrix (ECM) 136 (also referred to as ground substance) and encased in a synovial sheath 131. Ligament 110 has essentially the same basic structure, except without the synovial sheath. ECM 136 provides a sustaining and protective environment for collagen fibers 135, while synovial sheath 131 provides additional lubrication to facilitate movement of tendon 130.

ECM 136 within each tendon 130 (and carpal ligament 110) includes a proteoglycan (PG) component. This PG component plays a key role in the hydration of ECM 136, and in fact may be the cause of tendon swelling in CTS patients due to excess fluid retention. Consequently, the direct delivery of a dehydrating agent to the region of carpal tunnel 105 can in many cases provide immediate and significant reduction of swelling within flexor tendons 130 and/or carpal ligament 110 (and hence reduce compression of median nerve 120).

Accordingly, in FIG. 1C, a delivery system 150 is used to deliver a dehydrating agent 155 to the region of carpal tunnel 105. Note that while delivery system 150 is depicted as incorporating a needle 151 (such as would be attached to a syringe, pump, or any other external supply system) for exemplary purposes, in various other embodiments, delivery system 150 can incorporate any type of mechanism capable of delivering dehydrating agent 155 to the region around carpal tunnel 105, such as a needleless delivery system (e.g., compressed gas delivery or micro-needle patch), a temporary or permanent shunt, or an implant (e.g., timed release capsule or miniature pump), among others. In some embodiments, the use of controlled release systems such as pumps, patches, implants, and timed release capsules can beneficially allow parameters such delivery duration, delivery rate, and delivery frequency (i.e., delivery intervals) to be defined (before, during, and/or after application of the delivery system).

When performed as a minimally invasive procedure (e.g., using needle 151), fluoroscopic, ultrasonic, endoscopic, or other visualization or navigation methods can optionally be used to ensure the proper positioning and application needle 151, as well as monitor the actual delivery of dehydrating agent 155. However, in various other embodiments, the dehydrating agent delivery procedure can be performed as an open or mini open procedure via one or more incisions in the skin of the patient.

As noted above, dehydrating agent 155 would preferably act upon the PG component of the target structures (e.g., flexor tendons 130 and/or carpal ligament 110), without affecting the collagen elements of those (and surrounding) structures, thereby minimizing the risk of weakening or harming any of the local anatomical structures. In some embodiments, dehydrating agent 155 can include enzymes that degrade proteoglycans (e.g., chondroitinase ABC, matrix metalloproteinase-3, and hyaluronidase) and polycationic molecules (e.g., polylysine) that displace water from the negatively charged proteoglycans to shrink the target tissues. Note that this listing is not intended to be comprehensive, and various other dehydrating agents will be readily apparent.

In some embodiments, dehydrating agent 155 can further include a marking agent (e.g., a radiopacifier). Such a marking agent could then be monitored to visualize (e.g., via fluoroscopy) or sense the location of dehydrating agent 155 to ensure that desired delivery control is maintained.

In some embodiments, delivery system 150 can be used to deliver dehydrating agent 155 directly to one or more flexor tendons 130 and/or carpal ligament 110 (i.e., via injection directly into such structures). In various other embodiments, delivery, system 150 can deliver dehydrating agent 155 to the general region of carpal tunnel 105 (e.g., into the interstitial regions between flexor tendons 130 and median nerve 120). Dehydrating agent 155 can then provide the dehydrating effect from within those interstitial regions, or can enter ECM 136 of tendons 130 (or of ligament 110) via osmotic transport or any other mechanism.

In some embodiments, dehydrating agent 155 may be embedded or integrated in a carrier structure or matrix (e.g., a desiccant gel or powder, a sponge or sponge particles, or any other absorptive material or structure). For example, proteoglycan-targeting enzyme in a carrier matrix could be applied to the surface of sheath(s) 131 of tendon(s) 130 of interest, thereby allowing the enzyme to diffuse into and dehydrate ECM 136 of the tendon(s) 130.

In various embodiments, the localized dehydration procedure described with respect to FIG. 1C can be supplemented with local therapeutic substance delivery (before, after, and/or during delivery of dehydrating agent 155). For example, an anti-inflammatory drug, antibiotic, corticosteroid, pain medication, analgesic, anesthetic, relaxant, lubricant, anti-fibrotic agent, growth factor, nucleic acids, cells, and/or other therapeutic agent could be delivered in conjunction with dehydrating agent 155 to provide enhanced or broader symptom management. In various embodiments, the therapeutic substance can be a liquid, gel, or solid (including particulate compositions). In various other embodiments, the therapeutic substance may be short-acting or formulated in a device or delivery mechanism that enables extended release and/or controlled release.

In some embodiments, especially if dehydrating agent delivery is performed in an open procedure, the therapeutic substance delivery can be via direct swabbing or placement of a therapeutic substance release carrier (e.g., a drug-infused sponge or implantable pump) in the region of carpal tunnel 105. However, in various other embodiments, therapeutic substance delivery can be via needles to match similarly minimally invasive dehydrating agent delivery steps.

For example, in one embodiment, therapeutic substance delivery can be performed through the same delivery system(s) 150 used to deliver dehydrating agent 155. For example, as indicated in FIG. 1C, needle 151 can be coupled to an optional secondary supply 156 (indicated by dotted lines) for therapeutic substance delivery. Needle 151 can either be left in place or repositioned prior to therapeutic substance delivery.

In various other embodiments, separate dehydrating agent and therapeutic substance delivery instruments can be used (e.g., a separate needle(s) and supply unit(s)), or various integrated systems could be used (e.g., a double-barreled or coaxial needle construction). In various other embodiments, dehydrating agent and therapeutic agent delivery can occur at the same or different locations simultaneously or sequentially.

In any event, the reduction in the swelling of flexor tendon(s) 130 and/or carpal ligament 110 can result in significantly less squeezing of median nerve 120, as depicted in the exemplary post-procedure cross section A-A of hand 100 shown in FIG. 1D. As shown in the detail view of tendon 130 in FIG. 1D, the reduction of fluid buildup in ECM 136 results in a reduced overall size (outer diameter) of tendon 130. Consequently, the dehydrated flexor tendons 130 take up less space within carpal tunnel 105, median nerve 120 is no longer compressed against transverse carpal ligament 110, and the CTS-related discomfort associated with such compression can be reduced or eliminated. This decompression is provided without damaging transverse carpal ligament 110, and so does not result in the problematic side effects associated with conventional carpal tunnel release surgery.

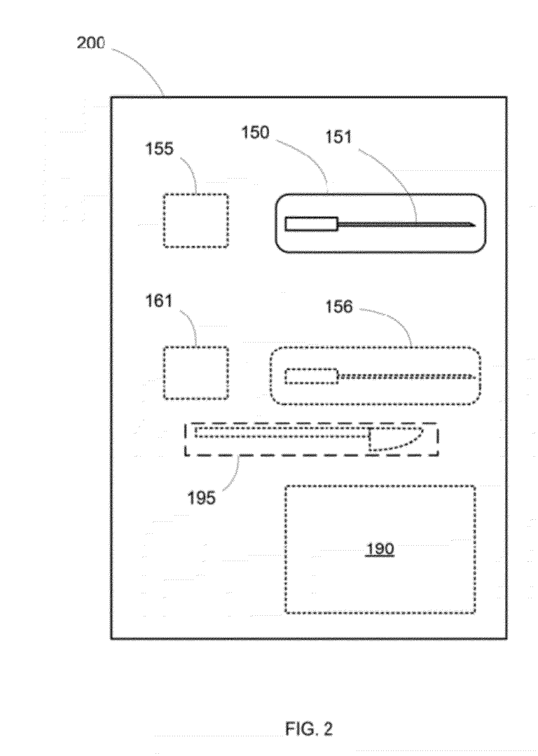

FIG. 2 shows an exemplary surgical kit 200 that includes a delivery system 150 (e.g., a needle 151) such as described above with respect to FIG. 1C, and an optional supply of dehydrating agent 155 (either in a separate storage container or preloaded into delivery system 150). Note that while a single delivery system 150 is depicted for clarity, kit 200 can include any number and types of delivery systems 150.

In various embodiments, kit 200 can further include instructions 190 that explain one or more uses of delivery system 150, such as described with respect to FIG. 1C. For example, in some embodiments, instructions 190 can describe placing needle 151 into or adjacent a tendon (e.g., flexor tendon 130) or ligament (e.g., carpal ligament 110), and delivering dehydrating agent 155 through needle 151. In various embodiments, instructions 190 can specify a maximum quantity of dehydrating agent 155 to be delivered to each tendon and/or ligament. In various other embodiments, instructions 190 can specify visual cues indicating proper placement of delivery system 150 at the tendon/ligament and/or appropriate swelling reduction of the tendon/ligament (either via direct visualization in an open procedure or via indirect visualization via endoscopic, ultrasonic, fluoroscopic, or navigation equipment).

In some embodiments, kit 200 can further include one or more secondary delivery systems 156 (e.g., needles) and/or therapeutic agents 161 (e.g., mixers, syringes, and/or vials/ampoules of drugs) for supplemental therapeutic substance delivery to the affected area. In such embodiments, instructions 190 can further specify the usage of secondary delivery system(s) 156 and/or therapeutic agent(s) 161 for therapeutic substance delivery in conjunction with the dehydrating agent delivery provided by delivery system 150, such as described with respect to FIG. 1C.

In various other embodiments, kit 200 can include optional additional instruments 195, such as cutting tools (e.g., a curette or scalpel), retractors, and/or mixers, among others. For example, additional instruments 195 could include a scalpel for creating an incision for allowing open access to the flexor tendons and/or carpal ligament for delivery of dehydrating agent 155 thereto.

While various embodiments of the invention have been described above, it should be understood that they have been presented by way of example only, and not limitation. Where methods and steps described above indicate certain events occurring in certain order, those of ordinary skill in the art having the benefit of this disclosure would recognize that the ordering of certain steps may be modified and that such modifications are in accordance with the variations of the invention. Additionally, certain steps may be performed concurrently in a parallel process when possible, as well as performed sequentially as described above. Thus, the breadth and scope of the invention should not be limited by any of the above-described embodiments, but should be defined only in accordance with the following claims and their equivalents. While the invention has been particularly shown and described with reference to specific embodiments thereof, it will be understood that various changes in form and details may be made.

1. A method for treating carpal tunnel syndrome in a patient, the method comprising delivering a dehydrating agent in or adjacent to a carpal tunnel in the patient,
wherein the carpal tunnel is partially defined by a carpal ligament,
wherein a first flexor tendon runs through the carpal tunnel, and
wherein the dehydrating agent reduces swelling of at least one of the first flexor tendon and the carpal ligament.

2. The method of claim 1, wherein the dehydrating agent is selected to cause proteoglycan dehydration without affecting collagen structures in the carpal tunnel.

3. The method of claim 2, wherein the dehydrating agent comprises at least one of a polycationic molecule and an enzyme that degrades proteoglycans.

4. The method of claim 2, wherein the dehydrating agent comprises at least one of chondroitinase ABC, matrix metalloproteinase-3, hyaluronidase, and polylysine.

5. The method of claim 1, wherein delivering the dehydrating agent comprises:
placing a needle in the first flexor tendon; and
conveying a first portion of the dehydrating agent through the needle into the first flexor tendon.

6. The method of claim 5, wherein delivering the dehydrating agent further comprises:
removing the needle from the first flexor tendon;
placing the needle in a second flexor tendon, wherein the second flexor tendon runs through the carpal tunnel; and
conveying a second portion of the dehydrating agent through the needle into the second flexor tendon.

7. The method of claim 5, wherein delivering the dehydrating agent further comprises:
placing a second needle in a second flexor tendon, wherein the second flexor tendon runs through the carpal tunnel; and
conveying a second portion of the dehydrating agent through the second needle into the second flexor tendon.

8. The method of claim 5, wherein delivering the dehydrating agent further comprises:
removing the needle from the first flexor tendon:
placing the needle in the carpal ligament; and
conveying a second portion of the dehydrating agent through the needle into the carpal ligament.

9. The method of claim 1, wherein delivering the dehydrating agent comprises conveying the dehydrating agent into the interstitial regions within the carpal tunnel.

10. The method of claim 1, wherein delivering the dehydrating agent comprises:
placing an implant adjacent to or within the carpal tunnel; and
conveying the dehydrating agent via the implant.

11. The method of claim 1, wherein delivering the dehydrating agent comprises conveying the dehydrating agent via a controlled release mechanism comprising at least one of a pump, a timed release capsule, and a patch.

12. The method of claim 1, further comprising delivering a therapeutic agent adjacent to or within the carpal tunnel, wherein the therapeutic agent comprises at least one of an anti-inflammatory drug, an antibiotic, a corticosteroid, a pain medication, an analgesic, an anesthetic, a relaxant, a lubricant, an anti-fibrotic agent, and a growth factor.

13. A kit comprising:
a delivery system; and
a set of instructions describing the use of the delivery system to deliver a dehydrating agent to a carpal tunnel region.

14. The kit of claim 13, wherein the delivery system comprises a first needle, and
wherein the set of instructions further describes placing the first needle into a first flexor tending running through the carpal tunnel region and conveying the dehydrating agent to the first flexor tendon via the first needle.

15. The kit of claim 14, wherein the set of instructions further describes placing the first needle into a second flexor tendon running through the carpal tunnel region and conveying the dehydrating agent to the second flexor tendon via the first needle.

16. The kit of claim 13, wherein the delivery system comprises an implant, and
wherein the set of instructions further describes at least one of placing the implant in the carpal tunnel region and placing the implant adjacent to the carpal tunnel region, and then having the implant to convey the dehydrating agent to the carpal tunnel region.

17. The kit of claim 13, wherein the delivery system comprises a controlled release system, and
wherein the set of instructions further describes at least one of a delivery duration, a delivery rate, and a delivery frequency for the controlled release system.

18. The kit of claim 17, wherein the controlled release system comprises at least one of a pump, a patch, and a timed release capsule.

19. The kit of claim 13, further comprising a supply of the dehydrating agent.

20. The kit of claim 19, wherein at least a portion of the supply of the dehydrating agent is preloaded into the delivery system.
